(19) United States
(12) Patent Application Publication
(10) Pub. No.: US 2021/0186982 A1
(43) Pub. Date: Jun. 24, 2021

(54) METHODS AND COMPOSITIONS FOR TREATING MELANOMA

(71) Applicants: UNIVERSITE NICE SOPHIA ANTIPOLIS, Nice (FR); INSERM (INSTITUT NATIONAL DE LA SANTE ET DE LA RECHERCHE MEDICALE), Paris (FR)

(72) Inventors: Marcel DECKERT, Nice Cedex 3 (FR); Sophie TARTARE - DECKERT, Nice Cedex 3 (FR); Aude MALLAVIALLE, Nice Cedex 3 (FR); Robin DIDIER, Nice Cedex 3 (FR)

(21) Appl. No.: 16/495,257

(22) PCT Filed: Mar. 23, 2018
(86) PCT No.: PCT/EP2018/057406

§ 371 Date: Sep. 18, 2019

(30) Foreign Application Priority Data
Mar. 24, 2017 (EP) 17305339.8

(51) Int. Cl. A61K 31/55; A61K 31/4025; A61K 31/45; A61P 35/04

(52) U.S. Cl. CPC A61K 31/55; A61P 35/04; A61K 31/45; A61K 31/4025

(57) ABSTRACT

The present invention relates to a method and composition for treating melanoma. More particularly, inventors have shown that high expression of USP14 correlates with melanoma progression and with a poorer survival rate in metastatic melanoma patients. Then, they have shown that an inhibition of ubiquitin-specific peptidase 14 (USP14) by siRNAs and pharmacological inhibitors (b-AP15, WP1130 and HBX41108), the cell proliferation of melanoma cell drastically decreased. They have also shown that melanoma treatment with pharmacological inhibitors can overcome resistance to drugs targeting oncogenic BRAF. Accordingly, the invention relates to a method for predicting the survival time of a subject suffering from melanoma by quantifying the expression level of USP14 in a biological sample and to a method of treating melanoma and resistant melanoma by using the inhibitors of USP14.

### FIELD OF THE INVENTION

The invention is in the field of oncology, more particularly the invention relates to a method and compositions for treating melanoma.

### BACKGROUND OF THE INVENTION

Cutaneous melanoma is a skin cancer whose incidence has increased dramatically over the last thirty years (http://www.who.int/uv/faq/skincancer/en/index 1.html) with more than 130,000 melanomas occurring worldwide each year. Although melanoma is the least common of skin cancer, it accounts for the vast majority of skin cancer death. Cutaneous melanoma is recognized for its propensity for early and extensive metastatic spread and seen as one of the most virulent and therapy resistant form of human cancers¹. These alarming facts raise skin melanoma to the ranks of the most aggressive skin cancers. Melanoma develops from melanocytes, cells originating from the neural crest located at the basal membrane of the epidermis¹. Melanoma progression is accompanied by driver mutations affecting the BRAF and NRAS genes (in 50% and 20% of melanoma, respectively) leading to constitutive activation of the MEK/ERK pathway. The mutation V600E is found in 90% of cases of BRAF mutant melanoma, currently making it a therapeutic target of choice. Other genetic and epigenetic changes, as well as the tumor microenvironment, affect the survival and proliferation of cancer cells and their metastatic ability by altering several signaling pathways². The majority of melanomas evolve in a first phase of horizontal growth (radial growth phase or RGP), followed by a second vertical phase during which cancer cells acquire new migratory and invasive properties that allow them to invade the dermis (Vertical growth phase or VGP).

Early stage melanomas can be successfully treated by surgical resection¹⁰. However, melanomas generally progress to metastatic forms resistant to radiotherapy and chemotherapy. New therapies such as immunotherapy (anti-CTLA4/PD1/PDL1) and targeted therapies (BRAF and/or MEK inhibitors) have led to improved survival in patients with metastatic disease2′ ¹¹. Since 2012, BRAF inhibitors (BRAFi) such as vemurafenib (PLX4032) are prescribed for the treatment of melanomas carrying the BRAF V600E mutation, with a remarkable response rate of 60%. Nevertheless, drug resistance invariably develops, and most patients progress within 6 to 12 months of treatment. Relapses are generally associated with acquired resistance linked to reactivation of the ERK pathway by secondary mutations of NRAS or MEK1, activation of tyrosine kinase receptors and PI3K/AKT and STAT3 survival pathways. Recent discoveries on the mechanisms of resistance to BRAFi have allowed the implementation of new therapeutic strategies, such as the combination of Dabrafenib (BRAFi) and Trametinib (MEKi). However, the long-term prognosis of metastatic melanoma is still very poor for most patients,

The enzymatic reaction that opposes the conjugation of ubiquitin by E3 ligases is the deubiquitination by deubiquitination enzymes (DeUBiquitinases or DUBs). DUBs represent ubiquitin-specific cysteine proteases, which can cleave one or more ubiquitin molecules on the target proteins, or even the entire poly-ubiquitin chain. The expression or abnormal activity of DUBs has been demonstrated in pathological situations, such as inflammation and cancer¹⁹. Some DUBs, such as USP14 (Ubiquitin-specific peptidase 14), are direct components of the 26S proteasome, thereby having a major impact on cellular proteostasis²⁰. DUBs are therefore promising therapeutic targets. However, with the exception of USP13 as a DUB of MITF 21 and USPS as a p53 protein regulator in melanomas in response to BRAF inhibitors²², the exact role of DUBs in the development of resistant metastatic melanoma remains poorly understood.

### SUMMARY OF THE INVENTION

The invention relates to a method for treating melanoma in a subject in need thereof comprising a step of administering said subject with a therapeutically effective amount of an inhibitor of USP14. In particular, the present invention is defined by the claims.

### DETAILED DESCRIPTION OF THE INVENTION

Firstly, inventors have shown that high expression of USP14 correlates with melanoma progression and with a poorer survival rate in metastatic melanoma patients. Secondly, inventors have shown that an inhibition of ubiquitin-specific peptidase 14 (USP14) by siRNAs and pharmacological inhibitors (b-AP15, WP1130 and HBX41108), the cell proliferation of melanoma cell drastically decreased. Then, they have shown that melanoma treatment with pharmacological inhibitors can overcome resistance to drugs targeting oncogenic BRAF. To validate the anti-melanoma activity of the inhibition of USP14 observed in vitro, they used a xenograft mouse model of melanoma development in which the BRAFi-resistant cell line were injected into nude mice. They have observed that tumor growth showed a marked decrease in melanoma burden in b-AP15-vs vehicle-treated mice. Finally, inventors have observed that USP14 controls melanoma viability independently of p53 and caspase proteolytic activities. Thus, these results reveals the proteasome-associated DUB USP14 as a novel and promising therapeutic target in melanoma.

### Method for Predicting the Survival Time of a Subject Suffering from Melanoma

In a first aspect, the invention relates to a method for predicting the survival time of a subject suffering from melanoma comprising the steps of i) quantifying the expression level of USP14 in a biological sample obtained from the subject; ii) comparing the expression level quantified at step i) with its predetermined reference value and iii) concluding that the subject will have a short survival time when the expression level of USP14 is higher than its predetermined reference value or concluding that the subject will have a long survival time when the expression level of USP14 is lower than its predetermined reference value.

The method is particularly suitable for predicting the duration of the overall survival (OS), progression-free survival (PFS) and/or the disease-free survival (DFS) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they have become cancer-free (achieved remission). DSF gives more specific information and is the number of people with a particular cancer who achieve remission. Also, progression-free survival (PFS) rates (the number of people who still have cancer, but their disease does not progress) include people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma, is a rare form of melanoma that occurs in the eye. In a particular embodiment, the melanoma is cutaneous melanoma.

As used herein, the term “subject” denotes a mammal, such as a rodent, a feline, a canine, and a primate. Particularly, the subject according to the invention is a human. More particularly, the subject according to the invention has or is susceptible to have melanoma. In particular embodiment, the subject has or is susceptible to have cutaneous melanoma. In a particular embodiment, the subject has or is susceptible to have metastatic melanoma.

As used herein, the term “USP14” refers to Ubiquitin-specific peptidase 14. USP14 is a protein that in humans is encoded by the USP14 gene. The naturally occurring human USP14 gene has a nucleotide sequence as shown in Genbank Accession number NM_001037334.1 and the naturally occurring human USP14 protein has an aminoacid sequence as shown in Genbank Accession number NP_001032411.1. The murine nucleotide and amino acid sequences have also been described (Genbank Accession numbers NM_001038589.2 and NP_001033678.1). USP14 belongs to deubiquitination enzymes family also known as DeUBiquitinases or DUBs. DUBs represent ubiquitin-specific cysteine proteases, which can cleave one or more ubiquitin molecules on the target proteins, or even the entire poly-ubiquitin chain. USP14 protein is located in the cytoplasm and cleaves the ubiquitin moiety from ubiquitin-fused precursors and ubiquitinylated proteins.

As used herein, the term “expression level” refers to the expression level of UPS14. Typically, the expression level of the USP14 gene may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

As used herein, the term “biological sample” refers to any sample obtained from a subject, such as a serum sample, a plasma sample, a urine sample, a blood sample, a lymph sample, or a tissue biopsy. In a particular embodiment, biological sample for the determination of an expression level include samples such as a blood sample, a lymph sample, or a biopsy. In a particular embodiment, the biological sample is a blood sample, more particularly, peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of

a) providing a collection of tumor tissue samples from subject suffering from melanoma;

b) providing, for each tumor tissue sample provided at step a), information relating to the actual clinical outcome for the corresponding subject (i.e. the duration of the disease-free survival (DFS) and/or the overall survival (OS));

c) providing a serial of arbitrary quantification values;

d) quantifying the cell density for each tumor tissue sample contained in the collection provided at step a);

e) classifying said tumor tissue samples in two groups for one specific arbitrary quantification value provided at step c), respectively: (i) a first group comprising tumor tissue samples that exhibit a quantification value for level that is lower than the said arbitrary quantification value contained in the said serial of quantification values; (ii) a second group comprising tumor tissue samples that exhibit a quantification value for said level that is higher than the said arbitrary quantification value contained in the said serial of quantification values; whereby two groups of tumor tissue samples are obtained for the said specific quantification value, wherein the tumor tissue samples of each group are separately enumerated;

f) calculating the statistical significance between (i) the quantification value obtained at step e) and (ii) the actual clinical outcome of the subjects from which tumor tissue samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 tumor tissue samples of 100 subjects. The 100 samples are ranked according to the cell density. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

### Method for Treating Melanoma

Inventors have shown that an inhibition of USP14 by siRNAs and pharmacological inhibitors, the cell proliferation of melanoma cell drastically decreased.

Accordingly, in a second aspect, the invention relates to a method for treating melanoma in a subject in need thereof comprising a step of administering said subject with a therapeutically effective amount of an inhibitor of USP14.

In a particular embodiment, the subject is identified as having a short survival time by performing the method as described above.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

The term “inhibitor of USP14” refers to a natural or synthetic compound that has a biological effect to inhibit the activity or the expression of USP14. More particularly, such compound by inhibiting USP14 activity induces a rapid accumulation of K48-linked poly-ubiquitinated proteins, the phosphorylation of the stress-related kinases p38 and JNK, and the up-regulation of the heat shock protein HSP70. The inhibition of USP14 triggers a potent ER stress response by the up-regulation of CHOP, BIP/GRP78, GADD34, ATF4, and the appearance of the spliced XBP1. In a particular embodiment, the inhibition of USP14 lead to a ROS-dependent, caspase-independent cell death associated with accumulation of poly-ubiquitinated proteins and chaperones, and ER stress.

In a particular embodiment, the inhibitor of USP14 is a peptide, petptidomimetic, small organic molecule, antibody, aptamers, siRNA or antisense oligonucleotide. The term “peptidomimetic” refers to a small protein-like chain designed to mimic a peptide. In a particular embodiment, the inhibitor of USP14 is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of USP14 is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da. In a particular embodiment, the small molecule is VLX1570 (phase I/II of clinical trial; developed by Vivolux). This small organic molecule has the formula C₂₃H₁₇F₂N₃O₆ and the following structure in the art:

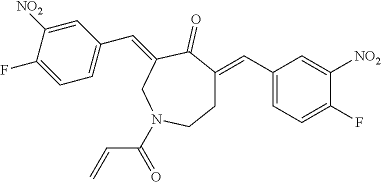 [SMILES: C=CC(=O)N1CC/C(=C\c2ccc(F)c([N+](=O)[O-])c2)C(=O)/C(=C/c2ccc(F)c([N+](=O)[O-])c2)C1]

In a particular embodiment, the small molecule is b-AP15. This small organic molecule has the formula C22H17N3O6 and the following structure in the art:

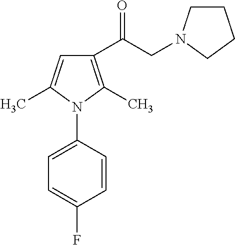 [SMILES: Cc1cc(C(=O)CN2CCCC2)c(C)n1-c1ccc(F)cc1]

In a particular embodiment, the small molecule is derivates of VLX1570 and b-AP15 as described in Wang et al Chem Biol Drug Des. 2015 November; 86(5): 1036-1048.

In a particular embodiment, the small molecule is IU1. This small organic molecule has the formula C18H21FN20 and the following structure in the art:

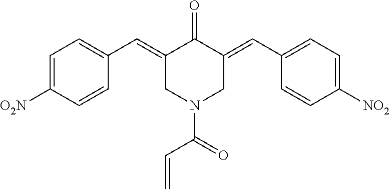 [SMILES: C=CC(=O)N1C/C(=C\c2ccc([N+](=O)[O-])cc2)C(=O)/C(=C/c2ccc([N+](=O)[O-])cc2)C1]

In some embodiments, the inhibitor of USP14 expression is a short hairpin RNA (shRNA), a small interfering RNA (siRNA) or an antisense oligonucleotide which inhibits the expression of USP14. In a particular embodiment, the inhibitor of USP14 expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of USP14 expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the inhibitor of USP14 is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the inhibitor is an intrabody having specificity for USP14. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

### Method for Treating Resistant Melanoma

Acquired resistance to targeted therapies is currently a clinical challenge in the treatment of advanced metastatic melanoma. Therefore, inventors examined the impact of targeting USP14 in melanoma cells resistant to BRAFV600E inhibitors (BRAFi). They have shown that melanoma treatment with pharmacological inhibitors against USP14 can overcome resistance to drugs targeting oncogenic BRAF.

Accordingly, in a further aspect, the invention relates to a method for treating resistant melanoma in a subject in need thereof comprising a step of administering said subject with a therapeutically effective amount of an inhibitor of USP14.

As used herein, the term “resistant melanoma” refers to melanoma which does not respond to a treatment. The cancer may be resistant at the beginning of treatment or it may become resistant during treatment. The resistance to drug leads to rapid progression of metastatic of melanoma. The resistance of cancer for the medication is caused by mutations in the gene which are involved in the proliferation, divisions or differentiation of cells. In the context of the invention, the resistance of melanoma is caused by the mutations (single or double) in the following genes: BRAF, MEK or NRAS. The resistance can be also caused by a double-negative BRAF and NRAS mutation.

In a particular embodiment, the melanoma is resistant to a treatment with the inhibitors of BRAF mutations. BRAF is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E.

The inhibitors of BRAF mutations are well known in the art. In a particular embodiment, the melanoma is resistant to a treatment with vemurafenib. Vemurafenib also known as PLX4032, RG7204 ou R05185426 and commercialized by Roche as Zelboraf. In a particular embodiment, the melanoma is resistant to a treatment with dacarbazine. Dacarbazine also known as imidazole carboxamide is commercialized as DTIC-Dome by Bayer. In a particular embodiment, the melanoma is resistant to a treatment with dabrafenib also known as tafinlar which is commercialized by Novartis.

In a further embodiment, the melanoma is resistant to a treatment with the inhibitors of MEK. MEK refers to Mitogen-activated protein kinase kinase, also known as MAP2K, MEK, MAPKK. It is a kinase enzyme which phosphorylates mitogen-activated protein kinase (MAPK). MEK is activated in melanoma. The inhibitors of MEK are well known in the art. In a particular embodiment, the melanoma is resistant to a treatment with trametinib also known as mekinist which is commercialized by GSK. In a particular embodiment, the melanoma is resistant to a treatment with cobimetinib also known as cotellic commercialized by Genentech. In a particular embodiment, the melanoma is resistant to a treatment with Binimetinib also knowns as MEK162, ARRY-162 is developed by Array Biopharma.

In a particular embodiment, the melanoma is resistant to a treatment with the inhibitors of NRAS. The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations. In a particular embodiment, the melanoma is resistant in which double-negative BRAF and NRAS mutant melanoma.

In a particular embodiment, the melanoma is resistant to a combined treatment. As used herein, the terms “combined treatment”, “combined therapy” or “therapy combination” refer to a treatment that uses more than one medication. The combined therapy may be dual therapy or bi-therapy. In the context of the invention, the melanoma is resistant to a combined treatment characterized by using an inhibitor of BRAF mutation and an inhibitor of MEK as described above. For example, the combined treatment may be a combination of vemurafenib and cotellic.

In a further embodiment, the melanoma is resistant to a treatment with an immune checkpoint inhibitor.

As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-C1-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

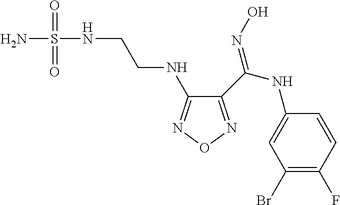 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

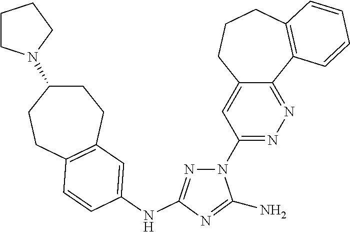 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “subject” denotes a mammal, such as a rodent, a feline, a canine, and a primate. Particularly, the subject according to the invention is a human. More particularly, the subject according to the invention has or susceptible to have melanoma. In a particular embodiment, the subject has or susceptible to have melanoma resistant to at least one of the treatments as described above. The subject having a melanoma resistant is identified by standard criteria. The standard criteria for resistance, for example, are Response Evaluation Criteria In Solid Tumors (RECIST) criteria, published by an international consortium including NCI.

As used herein the terms “administering” or “administration” refer to the act of injecting or otherwise physically delivering a substance as it exists outside the body (e.g., an inhibitor of USP14) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of inhibitor of USP14 for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitors of USP14 as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Method of Screening

A further object of the present invention relates to a method of screening a drug suitable for the treatment of melanoma comprising i) providing a test compound and ii) determining the ability of said test compound to inhibit the activity of USP14.

Any biological assay well known in the art could be suitable for determining the ability of the test compound to inhibit the activity of USP14. In some embodiments, the assay first comprises determining the ability of the test compound to bind to USP14. In some embodiments, a population of cells is then contacted and activated so as to determine the ability of the test compound to inhibit the activity of USP14. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of USP14, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1. Expression and activity of USP14 in melanoma cells. (A) Pharmacological inhibition of DUB activities reduces the proliferation of A375 cells. MTS assay on cells treated for 24 h with increasing doses of b-AP15, WP1130 and HBX41108 (0.1 to 10 μM) or 104 Staurosporine (STS) as control. (B) Principle of the in vitro labeling method of activated DUBs by the suicide substrate HA-Ub-VS (DUB trap assay). (C) Comparison of DUB activity between melanocytes (MHN) and melanoma cells using DUB trap assay. Lysates of the indicated cells were incubated at 37° C. with the HA-Ub-VS probe and analyzed by anti-HA and anti-USP14 immunoblots. The active form of USP14 is indicated (USP14-Ub-HA). (D) Inhibition of USP14 activity in A375 cells by increasing doses of b-AP15 measured by DUB trap assay and anti-USP14 immunoblot. (E) Inhibition of USP14 activity in 12051u, 501Mel, SKMe128 and 4511u cells by b-AP15 (2 μM) measured by DUB trap assay and anti-USP14 immunoblot. (F) Analysis of public bioinformatic dataset (GSE3189) associates USP14 gene expression with melanoma progression. (E) Survival curves combining a high level of USP14 gene expression with poor prognosis in metastatic melanoma. Data were collected from publicly available data set (GSE19234).

FIG. 2. USP14 inhibition has a potent and broad anti-melanoma effect. Inhibition of USP14 reduces the proliferation of 12051u, A375 and Me1501 cells. MTS assay on cells treated for 24 h with increasing doses of b-AP15 (0.01 to 10 μM).

FIG. 3. siRNA-mediated depletion of USP14 reduces melanoma cell survival. 12051u cells were transfected with 50 nM of siRNAs targeting USP14 (siUSP14 #1 and #2) or 50 nM of control siRNA for 3 days. The expression of USP14 and the phosphorylation of Rb were analyzed by Western blot after 5 days of transfection. Anti-HSP60 was used as a loading control.

FIG. 4. Characterization of molecular mechanisms underlying USP14 inhibition. (A) qRT-PCR analysis of mRNA levels of HSPA1A and ER stress response genes following incubation of 12051u cells with 2 μM b-AP15 for 6 h and 24 h. (B) USP14 inhibition triggers ROS production. A375 cells treated with 2 μM b-AP15 for 30 min in the presence or not of 5 mM NAC were stained with 10 μM CM-H2DCFDA for 30 min at 37° C. ROS levels were determined by flow cytometry. A treatment with H₂O₂ (10 μM) is shown as control.

FIG. 5. Treatment with b-AP15 overcomes resistance to BRAFV600E inhibitors. (A) Schematic description of the isogenic pairs of naive and BRAFi-resistant melanoma cells used in this study. (B) USP14 inhibition reduces the proliferation of m229 and 4511u BRAFi-sensitive cells and of their BRAFi-resistant derivatives m229R and 45 11uR, respectively. MTS assays on cells treated for 24 h with increasing doses of b-AP15 (0.1 to 10 μM). (C) Analysis of USP14 activity in m229/m229R and 4511u/4511uR pairs of melanoma cells using DUB trap assay. Lysates of the indicated cells were incubated at 37° C. with the HA-Ub-VS probe and analyzed by anti-HA and anti-USP14 immunoblots. The active form of USP14 is indicated (USP14-Ub-HA).

FIG. 6. b-AP15 inhibits tumor growth in melanoma xenografted mouse. (A) Schematic representation of the experimental procedure used in this study. (B) Quantification of tumor growth inhibition by b-AP15. Tumor BLIs of b-AP15- or vehicle-treated mice were recorded as described above and data analysed with the M3Vision software (Biospace Lab). Data shown are mean±SD of tumor BLI (n=12; **, p=0.009 (2way ANOVA).

FIG. 7. The b-AP15 derivative VLX1570 inhibits USP14 activity in melanoma cells. Analysis of USP14 activity in A375 cells treated with b-AP15 or VLX1570 using the DUB trap assay. Lysates were blotted with antibodies against USP14 and UCHL5. The active forms of USP14 and UCHL5 are indicated (USP14-Ub-HA and UCHL5-Ub-HA).

FIG. 8. USP14 inhibition induces growth inhibition and cytotoxicity on melanoma cell lines. (A) Growth curve of A375 treated with 2 μM b-AP15, 2 μM VLX1570 or 104 bortezomib (BTZ). Data were acquired in triplicate during 3 days using an IncuCyte Zoom. (B) and (C) Cytotoxic effects of USP14 inhibition on melanoma cells. A375 and Me1501 cells were treated with the indicated doses of b-AP15 or VLX1570, and stained with cytotox green reagent (100 nM). Cytotoxicity was monitored in triplicate for 72 h with an IncuCyte imaging system.

### EXAMPLE

Material & Methods

Cells and Reagents

Human melanoma cell lines were obtained as previously described 6, 8. The isogenic pairs of BRAFi-sensitive and BRAFi-resistant cells m229/m229R, m238/m238R and m249/m249R were obtained from Roger S. Lo (University of California, Los Angeles, USA). Patient melanoma cells (Pt.1, Pt.2 and Pt.3) were the kind gift of Robert Ballotti (Nice, France) and prepared as previously described 23. Melanoma cells were cultured in Dulbecco's modified Eagle Medium (DMEM) supplemented with 7% FBS (HyClone). Human primary epidermal melanocytes were isolated from foreskin and maintained as described previously 5. For in vivo bioluminescence imaging, 4511u-R-Luc+ cells were obtained by lentiviral transduction (pLenti6/V5-luciferase; Thermo Fischer Scientific, Waltham, Mass., USA) and blasticidin selection (2 μg/ml). All cell cultures were grown at 37° C. under 5% CO2

Primers and culture reagents were purchased from Thermo Fischer Scientific. The selective inhibitor of USP14 b-AP15, WP1130 and the JNK inhibitor SP600125 were from Merck Millipore (Darmstadt, Germany). Bortezomib (PS-341) was from Selleckchem (Houston, Tex.). The p38 MAP kinase inhibitor SB202190 (#1204) was from Tocris (Bristol, UK). QVD-OPH was from ApexBio Technology (Aurora, Ohio). Staurosporin, MG-132, N-Acetyl-L-cysteine (A8199), H₂O₂ and all other reagents were purchased from Sigma-Aldrich (St Louis, Mich., USA) unless otherwise stated.

Antibodies to USP14, HSP60, p53, p21, ERK2 (Santa Cruz Biotechnology), K48-linkage specific polyubiquitin (D9D5), JNK and phospho-JNK, p38 and phospho-p38 (Thr180/Tyr182), Pan AKT and P-AKT (ser473), phospho-MKK3 (Ser189)/MKK6 (Ser207), phospho-Rb (Ser807/Ser811) (Cell Signaling Technology), UCH37 (Bethyl Laboratories), K63-linkage specific Polyubiquitin (Abcam), Bip/GRP78 (BD biosciences)

Peroxidase-conjugated anti-rabbit antibodies were from Cell Signaling Technology. Peroxidase-conjugated anti-mouse and anti-goat antibodies were from Dako.

RNAi Studies

siRNAs were purchased from Dharmacon (Thermo Fisher Scientific). Transfection of siRNA was carried out using Lipofectamine RNAiMAX (Thermo Fisher Scientific) at a final concentration of 25 or 50 nM. Cells were assayed 3 or 5 days post transfection.

Cell Lysis and Immunoblot Analysis

Melanoma cells were harvested as described before 6. Cells were lysed at 4° C. in RIPA buffer (Millipore) supplemented with Pierce™ Protease and Phosphatase Inhibitor Mini Tablets, and briefly sonicated. Cell lysates were cleared at 16,000 xg for 15 min at 4° C. Whole cell lysates were subjected to SDS-PAGE and immunoblot analysis as previously described 6.

DUB Trap Assay

Cells were harvested, washed with PBS and pellets were dried then freezed (−80° C.) and subsequently lysed in ice-cold buffer containing 50 mM Tris (pH 7.4), 5 mM MgCl2, 250 mM sucrose, 1 mM DTT, 2 mM ATP, and 1 mM PMSF and mild sonication. Lysates were cleared by centrifugation, and 20 μg of protein extracts were incubated for 25 min at 37° C. with 2 μM HA-Ub-VS (Boston Biochem, Cambridge, Mass.). After boiling in reducing sample buffer, labelled cell lysates were subjected to immunoblot analysis as described above using the indicated antibodies.

Proteasome Activity Assay

Cells were stimulated with Bortezomib 2 μM or b-AP15 (1/2/5 μM) for 6 h. Cells were then collected, washed, and lysed for 30 min at 4° C. in a ATP-containing lysis buffer (50 mM HEPES pH 7.8, 5 mM ATP, 0.5 mM DTT, 5 mM MgCl2 and 0.2% Triton X-100). Cell lysates were cleared at 16,000 xg for 15 min at 4° C. Equal amounts of protein from each sample (10 μg/condition) were incubated in 96-well plates with 0.1 mM of Suc-Leu-Leu-Val-Tyr-AMC fluorogenic substrate (Enzo Life Sciences, Farmingdale, N.Y., USA) to measure chymotrypsin-like activities. Fluorescence intensity was measured during 2 h by following emission at 460 nm (excitation at 390 nm). Epoxomycin (100 nM) was used as a control inhibitor of chymotrypsin-like activity.

Proliferation Assays

Cell proliferation was measured by a MTS conversion assay using the CellTiter 96® Aqueous Non-Radioactive Cell Proliferation kit (Promega, Madison Wis.) according to the manufacturer instructions. Cells were seeded in 96-well plates (5×104 cells/well) and treated with different reagents for the indicated times and incubated at 37° C. with MTS reagent. The optical density of each sample at 490 nm was determined with a Multiskan FC plate reader (Thermo Fisher Scientific, Waltham, Mass. USA).

Alternatively, cell growth was assessed by crystal violet staining on cells seeded in 24-well plates for the indicated time. After treatment, cells were fixed in PFA 3% during 20 min, washed with PBS 3 times and stained with crystal violet 0.4% in ethanol 20% for 30 min.

Analysis of Apoptosis and Cell Cycle by Flow Cytometry

Cell cycle profiles and sub-G1 analysis were performed by flow cytometry analysis of propidium iodide (PI)-stained cells. Briefly, following permeabilization in icecold ethanol 70%, cells were stained with PI 40 μg/ml in PBS supplemented with RNAse A 100 μg/ml before analysis using a BD FACSCanto cytometer.

Cell death was evaluated by flow cytometry following staining with Annexin-V-FITC and 7-AAD (eBiosciences) as previously described 24.

Measurement of ROS Production

ROS levels were measured using the redox-sensitive dye CM-H2DCFDA (Thermo Fischer Scientific). After treatment, cells were stained with 10 μM CM-H2DCFDA in PBS for 30 min at 37° C. Cells were washed with PBS and resuspended in PBS 5 mM EDTA/1% BSA. ROS production was analysed using a MACSQuant® Analyzer 10 cytometer (Miltenyi Biotech).

Immunofluorescence Analysis

Melanoma cells were grown to confluence on glass coverslips. After the indicated treatments, cell monolayers were rinsed briefly in PBS, fixed in 4% formaldehyde for 15 min, washed and permeabilized with 0.1% Triton X-100 in PBS for 2 min. After another washing with PBS, cells were incubated with anti-HSP70 antibody diluted in PBS, 1% BSA for 1 h. Cells were then washed and incubated with secondary anti-goat antibody. Cells were washed, incubated with Alexa Fluor-conjugated secondary antibodies (ThermoFischerScientific) and mounted in ProLong Gold Antifade Reagent with DAPI (Life Technologies). Images were captured on a Zeiss LSM 510 META laser scanning confocal microscope (Zeiss, Germany).

Real Time Quantitative PCR

Total RNA was extracted from cell samples using Nucleospin RNAII kit (Macherey-Nagel) and following the manufacturer's instructions. Recovered RNA samples were quantified using NanoDrop spectrophotometer ND1000 (Thermo Fisher Scientific). Reverse transcription was performed on 1 μg of total RNA in a volume of 20 μL using High capacity cDNA Reverse Transcription kit (Applied Biosystems) according to the manufacturer's instructions. Quantitative PCR was performed on 20 ng cDNA samples, in sealed 96-well microtiter plates using the Platinum® SYBR Green qPCR Supermix-UDG w/ROX (Life Technologies) with the StepOnePlus System (Applied Biosystems). Relative mRNA levels were determined using the 2ΔΔCT method and ACTB and PPIA as housekeeping genes. Values are the mean of duplicates and are representative of two independent experiments.

Gene Expression Analysis from Human Databases

Publicly available gene expression data sets from Gene Expression Omnibus (GEO) database were used to analyse USP14 levels in melanoma progression (GSE3189) and patient outcome (GSE19234). Normalized data were analyzed using GraphPad Prism (GraphPad software, San Diego, USA).

In Vivo Experiments

All mouse experiments were carried out in accordance with the Institutional Animal Care and the local ethics committee. For human melanoma xenografts, 5-week-old female athymic (nu/nu) mice (Harlan) were subcutaneously injected with 1×106 451Lu-R BRAF inhibitor resistant melanoma cells engineered to express a luciferase reporter (451Lu-R Luc+ cells) in 100 μl of PBS. After 3 days, mice were injected every 3 days intraperitoneally with vehicle or 10 mgkg-1 b-AP15 in 90/1/9 mix of Labrafil/Tween 80/DMA. At the indicated time, mice were anesthetized and injected intraperitoneally with 50 mgkg-1 D-luciferin (Perkin Elmer) in PBS. Images were acquired using a Photon Imager (Biospace Lab) system and data analysed with the M3Vision software (Biospace Lab). Tumor growth was monitored and quantified using BLI. The total numbers of photons per second per steradian per square centimeter were recorded. For BLI plots, photon flux was calculated for each mouse by using a rectangular region of interest encompassing the thorax of the mouse in a prone position. This value was normalized to the value obtained immediately after injection (15 min), so that all mice had an arbitrary starting BLI signal of 100.

Statistical Analysis

Unless otherwise stated all experiments were repeated at least three times and representative data/images are shown. Statistical analysis was performed using the Prism V5.0b software (GraphPad, La Jolla, Calif., USA). All data are presented as mean±SEM. For comparisons between two groups, P values were calculated using unpaired one-sided t-test or Mann-Whitney test. Statistical significance of the in vivo experiment was calculated with the two-way ANOVA test. P values of 0.05 (*), 0.01 (**) and 0.001 (***) were considered statistically significant.

TABLE 1
b-AP15 has a potent anti-melanoma effect irrespective of mutational
status, transcriptional background and acquired drug resistance.
IC50 (μm) of b-AP15 treatment on melanoma cell proliferation
was determined after 48 h by a MTS conversion assay.

| | | | | b-AP15 |
|---|---|---|---|---|
| | | | | IC50 |
| Cell line | Type | Mutation(s) | Resistance | (μM) |
| SBCL2 | RGP | NRASQ61K | none | 0.4 |
| WM793 | VGP | BRAFV600E/PTEN* | none | 0.3 |
| HMV2 | MET | NRAS* | none | 1.8 |
| WM164 | MET | BRAFV600E/CDKN2A* | none | 0.9 |
| WM266-4 | MET | BRAFV600D/PTEN* | none | 0.5 |
| MeWo | MET | p53*/CDKN2A* | none | 0.7 |
| 501Mel | MET | BRAFV600E | none | 0.5 |
| 1205Iu | MET | BRAFV600E/PTEN* | none | 0.4 |
| 451Iu | MET | BRAFV600E/p53* | none | 0.9 |
| 451IuR | MET | BRAFV600E/p53*/? | dabrafenib | 1.3 |
| m229 | MET | BRAFV600E/PTEN* | none | 1.2 |
| m229R | MET | BRAFV600E/PTEN*/RTK* | vemurafenib | 1.1 |
| m238 | MET | BRAFV600E/PTEN* | none | 0.4 |
| m238R | MET | BRAFV600E/PTEN*/RTK* | vemurafenib | 0.2 |
| m249 | MET | BRAFV600E/PTEN* | none | 2.0 |
| m249R | MET | BRAFV600E/PTEN*/NRAS* | vemurafenib | 3.0 |
| Mel1617 | MET | BRAF*/p53* | none | 1.4 |
| Mel1617R | MET | BRAF*/p53*/? | dabrafenib | 0.9 |
| A375 | MET | BRAFV600E/CDKN2A* | none | 0.6 |
| A375DR | MET | BRAFV600E/CDKN2A*/? | vemurafenib/ | 1.3 |
| | | | ERKi | |

| | | | | b-AP15 |
|---|---|---|---|---|
| | | | | IC50 |
| Patient cells | Type | Mutation(s) | resistance | (μM) |
| Pt#1 | MET | BRAF* | none | 3.4 |

*gene mutation or alteration; RGP, radial growth phase; VGP, vertical growth phase; MET, metastasis; RTK, Receptor tyrosine kinases.

Results

Expression and Activity of USP14 in Melanoma Cells

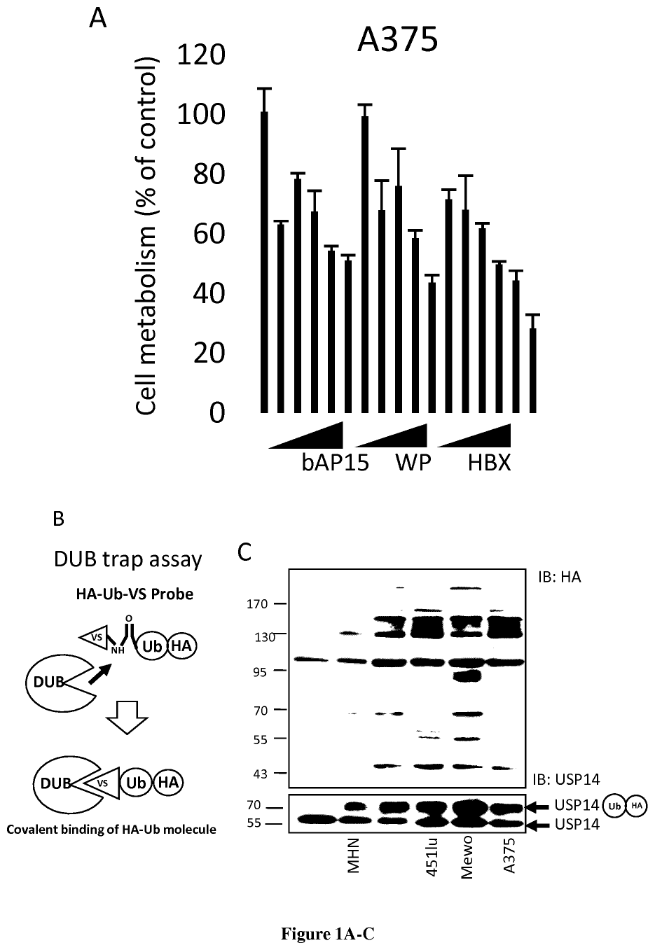

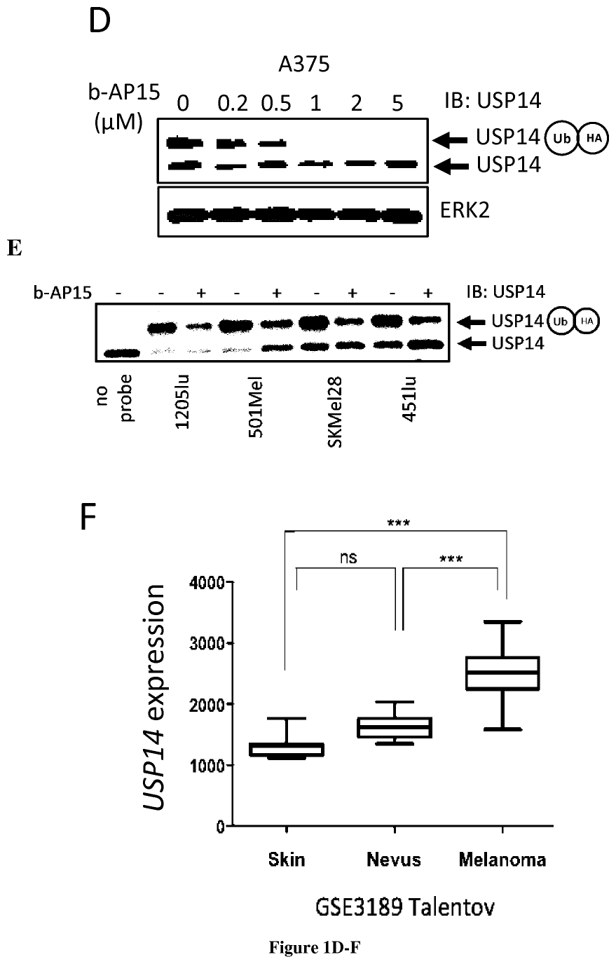

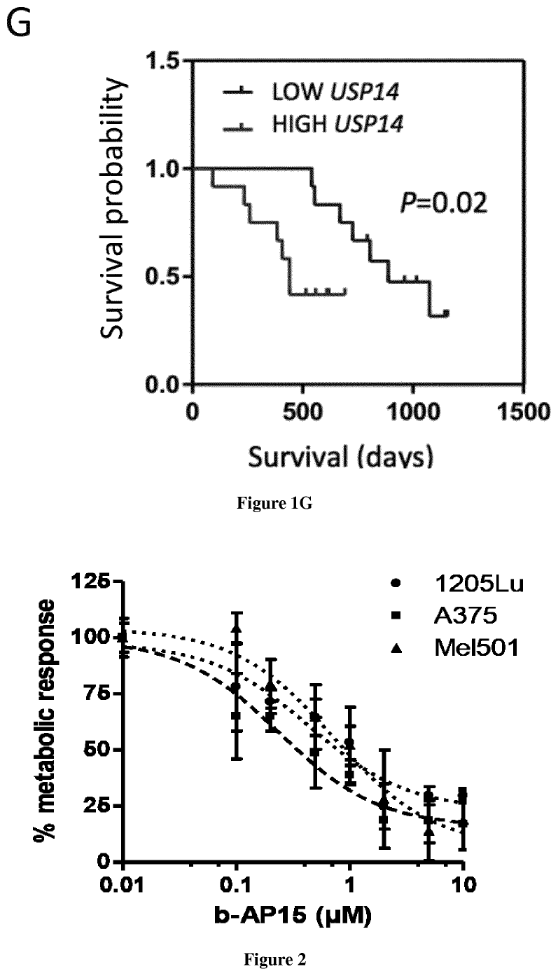

Increasing evidence points to an important role of DUBs in cancer 25. We thus investigated the involvement of these enzymes in cutaneous melanoma biology. Using the pharmacological inhibitors of DUB activity, b-AP15, WP1130 and HBX41108, we found that blocking DUB activity drastically decreased cell proliferation of A375 melanoma cell culture in a dose-dependent manner (FIG. 1A). To further identify DUBs that may be involved in melanoma biology, we have developed a DUB trap assay with the probe HA-Ub-VS, which covalently label with a HA-tagged ubiquitin molecule active DUBs in cell lysates 26 (FIG. 1B). DUB trap assays performed on melanocyte and melanoma cell lysates revealed an increased activity of several DUBs in melanoma cells compared to melanocytes. Given the anti-melanoma activity of b-AP15, which has been described as an inhibitor of the DUB USP1427, we thus assessed the activity of USP14 in melanoma. Anti-USP14 immunoblot analysis of the above DUB trap assays showed that compared to melanocytes, USP14 activity is significantly increased in melanoma cell lines 501Mel, 4511u, MeWo and A375 (FIG. 1C). We next confirmed that b-AP15 efficiently blocked the activity of USP14 in a dose-dependent manner in A375 melanoma cells (FIG. 1D), and in melanoma cell lines 12051u, 501Mel, SKMe128 and 4511u (FIG. 1E). Consistent with the effect of b-AP15, VLX1570, a novel small-molecule inhibitor of USP14 (37, 38) also blocked USP14, but not UCHL5, activity (FIG. 7). In order to confirm USP14 as a potential target in melanoma, we assessed USP14 gene expression in annotated NCBI Gene Expression Omnibus (GEO) datasets comparing patients tumors with their normal tissue counterparts and benign tumors. While USP14 levels were not significantly increased between normal skin samples and nevi, its expression was statistically increased in melanomas when compared to both normal skin and nevi (FIG. 1F). Interestingly, further analysis of public databases associates high USP14 expression with a lower probability of survival in patients with metastatic melanoma (FIG. 1G). USP14 inhibition has a potent and broad anti-melanoma effect

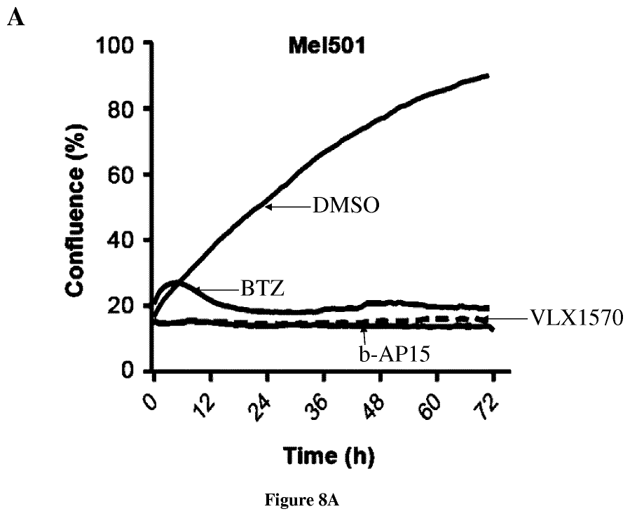

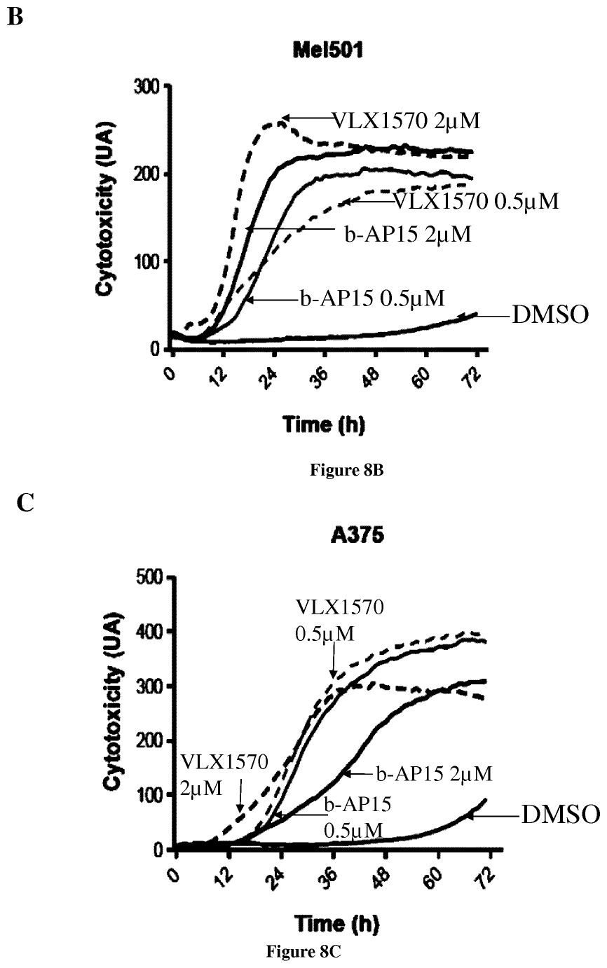

To study the role of USP14 in melanoma, we first examined the impact of USP14 inhibitor b-AP15 on a collection of melanoma cell lines with diverse mutational status. These cell lines are also representative of the three major subtypes of melanoma based on the frequency of BRAF and NRAS mutations: mutant BRAF, mutant RAS and wild-type. The incubation of 1205Lu, 501me1 and A375 cells with b-AP15 during 24 h impaired cell proliferation in a dose-dependent manner (FIG. 2). Using the same approach, we calculated the IC50 of b-AP15 for 16 melanoma cell lines to be in a μM range (0.4 to 2 μM) (Table 1). Our data show that pharmacological inhibition of USP14 by b-AP15 similarly reduced proliferation of mutant BRAF, mutant RAS and wild-type melanoma cells. Treatment with b-AP15 also blocks the proliferation of melanoma cell irrespective of the mutational status of TP53, PTEN or CDKN2A and of their transcriptional cell state. Real-time imaging further shows that the effect of b-AP15 on cell proliferation was rapid and compared to what was observed with the proteasome inhibitor bortezomib or with VLX1570 (FIG. 8A). We next evaluated how b-AP15 affects one of the characteristic property of tumor cells, which is the ability of cells to proliferate in colonies. Me1501 and A375 cells treated with b-AP15 are no longer capable of forming colonies when isolated, compared to untreated cells. The cytotoxic effect of b-AP15 is rapid, resulting in a rounding of melanoma cells within 12 h of treatment (data not shown). Cell cycle analysis performed on melanoma cells treated or not with b-AP15 for 24 h revealed that USP14 inhibition, which slightly modified cell cycle progression, massively increased cell death as indicated by the appearance of a sub-G1 cell population with reduced DNA content (19% and 11% for 12051u and A375 cells, respectively) (data not shown). For comparison, BRAF inhibitor Vemurafenib (PLX4032) completely arrested cells in the G0/G1 phase of the cell cycle. To confirm the effect of b-AP15 on melanoma cell death, we performed a flow cytometric analysis of Annexin-V-FITC/7-AAD labeling of A375 cells treated or not with b-AP15 (data not shown). Data showed a significant reduction in the viability of A375 cells exposed to b-AP15 (52%) compared to solvent effect (87%), with increase in both early and late apoptotic population. As a control, treatment with the cell death inducer staurosporine led to a similar decrease in cell viability (56%). At the molecular level, immunoblot analysis showed that USP14 targeting in 12051u and A375 cells altered the expression or phosphorylation of proteins related to cell proliferation and apoptosis. Treatment with b-AP15 increased levels of p53 and of the cell cycle inhibitor p21, while reducing the levels of phosphorylated Rb proteins. After 24 h of treatment, the drug caused the appearance of active fragments of caspase 3 and the cleavage of its nuclear substrate PARP (data not shown). Importantly, melanoma cell death was induced to similar levels following incubation with VLX1570 (FIGS. 8B and C). Together, these observations demonstrate that inhibition of USP14 by b-AP15 and VLX1570 has a potent anti-melanoma effect irrespective of mutational status of oncogenes and tumor suppressor and of transcriptional background.

Depletion of USP14 in Melanoma Reduces Cell Survival

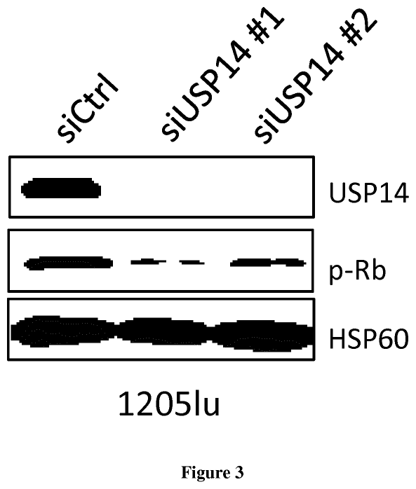

We next used a genetic approach based on small interfering RNAs (siRNAs) targeting USP14 to study the effects of USP14 depletion on melanoma cell survival. Two siRNA sequences (siUSP14 #1 and #2) and one control siRNA directed against luciferase (siLuc) were transfected into 12051u cells for 3 days. Immunoblot analysis shows that siUSP14 #1 and #2 efficiently decreased USP14 expression and similarly decreased phosphorylation of Rb proteins (FIG. 3). We then studied the impact of USP14 depletion on melanoma clonogenic cell growth. While Me1501 and A375 cells transfected with control siRNA (siLuc) formed colonies after 7 days, cells transfected with siUSP14 #2 are no longer capable of forming colonies (data not shown). Consistent with this, cell cycle analysis performed on USP14-depleted 12051u cells showed that suppression of USP14 for 6 days altered cell cycle progression and massively increased cell death as indicated by the appearance of a sub-G1 cell population with reduced DNA content (data not shown). Induction of cell death caused by USP14 knockdown in melanoma cells was further confirmed on USP14-depleted cells stained by Annexin-V-FITC/7-AAD (data not shown) and by immunoblot analysis revealing that USP14 depletion, which reduced Rb phosphorylation, also led to the cleavage of caspase 3 and its substrate PARP, two markers of apoptosis (data not shown). These data confirm that USP14 is an important regulator of melanoma cell survival.

Molecular Mechanisms Underlying USP14 Inhibition

In order to clarify how UPS14 regulates melanoma cell survival, we first assessed the importance of p53 and caspase-mediated apoptotic process in the cytotoxicity of b-AP15. Cell cycle analysis by flow cytometry of 12051u cells transfected 6 days with siLuc, siUSP14 #2 alone or siUSP14 #2 in combination with a siRNA directed against p53 (sip53) showed that p53 knockdown did not not prevent cell death induced by USP14 depletion (data not shown). Our data also indicated that melanoma cell death induced by b-AP15 took place independently of caspase activity. Surprisingly, the blockade of caspases by the pan-caspase inhibitor QVD-OPh did not prevent the cytotoxic action of b-AP15, although it fully prevented the cleavage of caspase 3 and PARP induced by b-AP15 treatment (data not shown). These data suggest that USP14 controls melanoma viability independently of p53 and caspase proteolytic activities.

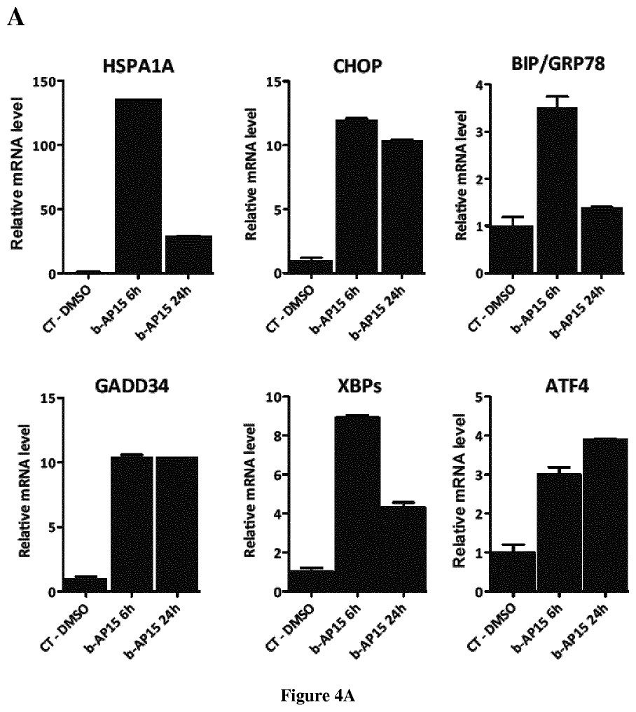

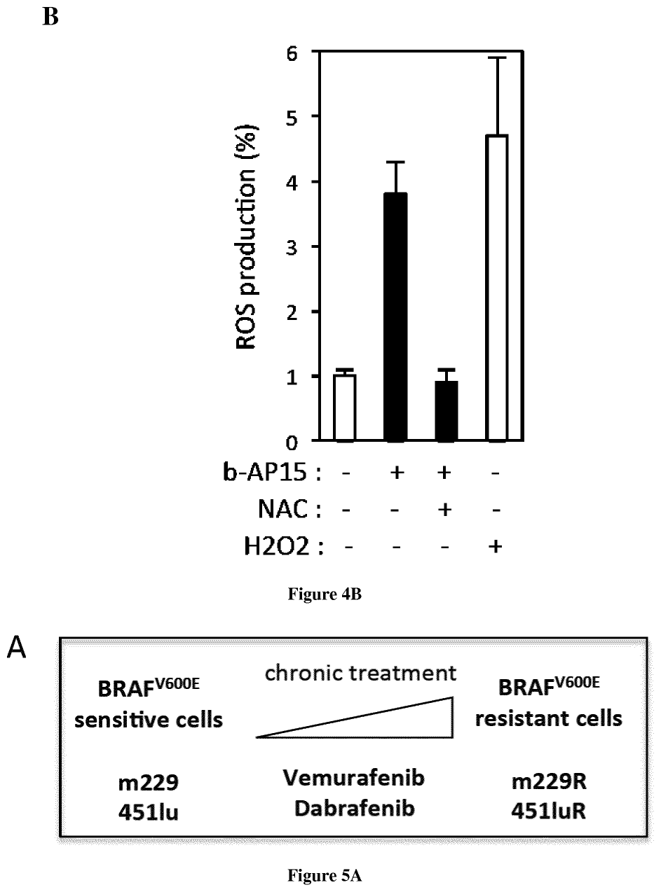

USP14 is a DUB predominantly associated with the proteasome 20, where it cleaves the poly-ubiquitin chains of proteins addressed to the proteasome 28. In myeloma cells, USP14 inhibition has been shown to trigger the accumulation of poly-ubiquitinated proteins and an ER stress response 29. We therefore examined how USP14 inhibition affects these events in melanoma cells. Treatment of A375 cells with b-AP15 induced a rapid accumulation of K48-linked poly-ubiquitinated proteins, the phosphorylation of the stress-related kinases p38 and JNK (FIG. 4C), and the up-regulation of the heat shock protein HSP70 (FIG. 4A). Using qRT-PCR analysis, we further determined that USP14 inhibition in melanoma cells triggered a potent ER stress response as shown by the up-regulation of CHOP, BIP/GRP78, GADD34, ATF4, and the appearance of the spliced XBP1 (data not shown). The induction of an ER stress response signature following USP14 inhibition in melanoma cells was confirmed by immunoblot analysis (data not shown). Finally, flow cytometry analysis revealed that treatment of melanoma cells with b-AP15 triggered a rapid burst of ROS, that is inhibited by the ROS scavenger N-acetyl-L-cysteine (NAC) (FIG. 4B). Consistent with this, the cytotoxicity of b-AP15 was blocked by incubating melanoma cell cultures with NAC, but not by the pan-caspase inhibitor QVD-Oph (FIG. 4F). Our data indicate that targeting USP14 lead to a ROS-dependent, caspase-independent cell death associated with accumulation of poly-ubiquitinated proteins and chaperones, and ER stress.

Targeting USP14 Overcomes Resistance to BRAFV600E Inhibitors

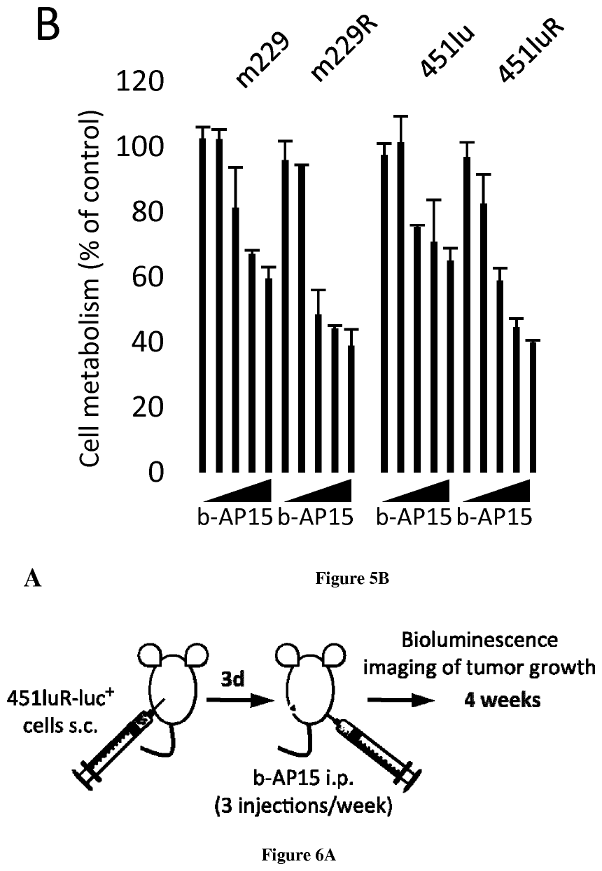

Acquired resistance to targeted therapies is currently a clinical challenge in the treatment of advanced metastatic melanoma. Therefore, we examined the impact of targeting USP14 in melanoma cells resistant to BRAFV600E inhibitors (BRAFi). We first used two resistant cell lines that were isolated from parental BRAFV600E melanoma cells following chronic treatment with vemurafenib (isogenic pair m229 and m229R) or dabrafenib (isogenic pair 4511u and and 4511uR) (FIG. 5A) 30. The inhibition of USP14 with b-AP15 potently decreased cell proliferation of BRAFi-resistant cells m229R and 4511uR, in a range of concentration that was comparable to the parental BRAFi-sensitive cells m229 and 4511u (FIG. 5B). Consistent with this, a DUB trap assay performed on lysates from the two pairs of cells showed that USP14 activity was not significantly different between BRAFi-sensitive and BRAFi-resistant cells (FIG. 5C). We extended these observations to additional isogenic pairs of cells sensitive and resistant to BRAFi, and using cell proliferation assays, we calculated the IC50 of b-AP15 on sensitive and BRAFi-resistant cells (Table 1). We found that b-AP15 blocked proliferation of vemurafenib- or dabrafenib-resistant cells with an efficacy not significantly different to what is observed on the respective parental BRAFi-sensitive cells. Notably, BRAFi-resistant cells were efficiently targeted by b-AP15 regardless of the molecular mechanisms of acquired resistance. In addition, USP14 inhibition efficiently decreased the viability of A375 melanoma cells that we have selected to acquire resistance to both BRAF and ERK inhibition (A375DR cells) (Table 1). A colony formation assay carried out on 4511u and 4511uR cells further confirmed that b-AP15 could suppressed melanoma colony formation independently of acquired resitance to BRAFi (data not shown). Mechanistically, inhibition of USP14 in melanoma cells induced molecular events, including accumulation of poly-ubiquitination, decreased Rb phosphorylation, increased p38 phophorylation and ER stress response, that were indistinguishable between BRAFi-sensitive and BRAFi-resistant cells (FIG. 5C). Our data show that melanoma treatment with b-AP15 can overcome resistance to drugs targeting oncogenic BRAF.

Anti-tumor activity of b-AP15 in a pre-clinical mouse model of melanoma

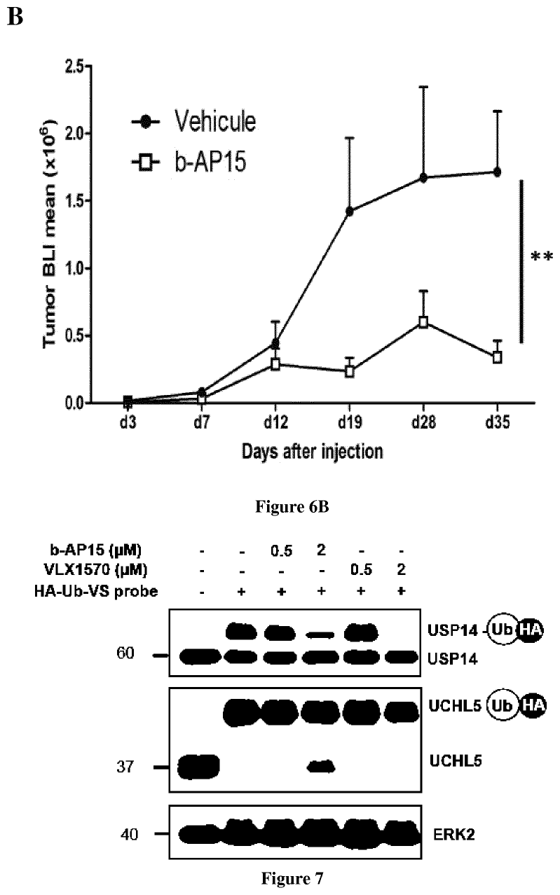

To further validate the anti-melanoma activity of b-AP15 observed in vitro, we used a xenograft mouse model of melanoma development in which the BRAFi-resistant cell line 4511u-R stably expressing the luciferase gene (451Lu-R Luc+ cells) were injected subcutaneously into nude mice (FIG. 6A). After 3 days, mice were divided into two groups: one group was injected i.p with b-AP15, while the other group was injected with vehicle alone. Bioluminescence analysis of tumor growth showed a marked decrease in melanoma burden in b-AP15-vs vehicle-treated mice (data not shown). The effect of b-AP15 treatment was already observable after 10 days and maintained up to day 35 of the experiment, as evidenced by bioluminescence imaging and the measurement of the tumor size at end point (FIG. 6B). Importantly, the doses of b-AP15 received by mice were well tolerated, since no weight loss was observed during the course of the study (data not shown). Our data thus reveal a potent in vivo anti-melanoma activity of b-AP15 and further suggest that targeting USP14 could represent a novel tool to treat melanoma that have acquired resistance to targeted therapy.

### REFERENCES

Throughout this application, various references describe the state of the art to which this invention pertains. The disclosures of these references are hereby incorporated by reference into the present disclosure.
1. Miller, A. J. & Mihm, M. C., Jr. Melanoma. N Engl J Med 355, 51-65 (2006).
2. Flaherty, K. T., Hodi, F. S. & Fisher, D. E. From genes to drugs: targeted strategies for melanoma. Nat Rev Cancer 12, 349-361 (2012).
3. Nieto, M. A., Huang, R. Y., Jackson, R. A. & Thiery, J. P. Emt: 2016. Cell 166, 21-45 (2016).
4. Gaggioli, C. et al. HGF induces fibronectin matrix synthesis in melanoma cells through MAP kinase-dependent signaling pathway and induction of Egr-1. Oncogene 24, 1423-1433 (2005).
5. Robert, G. et al. SPARC represses E-cadherin and induces mesenchymal transition during melanoma development. Cancer Res 66, 7516-7523 (2006).
6. Fenouille, N. et al. The Epithelial-Mesenchymal Transition (EMT) Regulatory Factor SLUG (SNA12) Is a Downstream Target of SPARC and AKT in Promoting Melanoma Cell Invasion. PLoS One 7, e40378 (2012).
7. Puisieux, A., Brabletz, T. & Caramel, J. Oncogenic roles of EMT-inducing transcription factors. Nat Cell Biol 16, 488-494 (2014).
8. Tichet, M. et al. Tumour-derived SPARC drives vascular permeability and extravasation through endothelial VCAM1 signalling to promote metastasis. Nature communications 6, 6993 (2015).
9. Kemper, K., de Goeje, P. L., Peeper, D. S. & van Amerongen, R. Phenotype switching: tumor cell plasticity as a resistance mechanism and target for therapy. Cancer Res 74, 5937-5941 (2014).
10. Clark, W. H., Jr. et al. Model predicting survival in stage I melanoma based on tumor progression. Journal of the National Cancer Institute 81, 1893-1904 (1989).
11. Eggermont, A. M., Spatz, A. & Robert, C. Cutaneous melanoma. Lancet 383, 816-827 (2014).
12. Balch, W. E., Morimoto, R. I., Dillin, A. & Kelly, J. W. Adapting proteostasis for disease intervention. Science 319, 916-919 (2008).
13. Deshaies, R. J. Proteotoxic crisis, the ubiquitin-proteasome system, and cancer therapy. BMC biology 12, 94 (2014).
14. Hershko, A. & Ciechanover, A. The ubiquitin system. Annu Rev Biochem 67, 425-479 (1998).
15. Finley, D. Recognition and processing of ubiquitin-protein conjugates by the proteasome. Annu Rev Biochem 78, 477-513 (2009).
16. Hu, R. & Aplin, A. E. Skp2 regulates G2/M progression in a p53-dependent manner. Mol Biol Cell 19, 4602-4610 (2008).
17. Aydin, I. T. et al. FBXW7 mutations in melanoma and a new therapeutic paradigm. Journal of the National Cancer Institute 106, djul07 (2014).
18. Fenouille, N. et al. The p53/p21(Cip1/Waf1) pathway mediates the effects of SPARC on melanoma cell cycle progression. Pigment Cell Melanoma Res 24, 219-232 (2011).
19. Nijman, S. M. et al. A genomic and functional inventory of deubiquitinating enzymes. Cell 123, 773-786 (2005).
20. D'Arcy, P. & Linder, S. Proteasome deubiquitinases as novel targets for cancer therapy. Int J Biochem Cell Biol 44, 1729-1738 (2012).
21. Zhao, X., Fiske, B., Kawakami, A., Li, J. & Fisher, D. E. Regulation of MITF stability by the USP13 deubiquitinase. Nature communications 2, 414 (2011).
22. Potu, H. et al. Usp5 links suppression of p53 and FAS levels in melanoma to the BRAF pathway. Oncotarget 5, 5559-5569 (2014).
23. Cerezo, M. et al. Metformin blocks melanoma invasion and metastasis development in AMPK/p53-dependent manner. Mol Cancer Ther 12, 1605-1615 (2013).
24. Baudot, A. D. et al. The tyrosine kinase Syk regulates the survival of chronic lymphocytic leukemia B cells through PKCdelta and proteasome-dependent regulation of Mc-1 expression. Oncogene 28, 3261-3273 (2009).
25. Fraile, J. M., Quesada, V., Rodriguez, D., Freije, J. M. & Lopez-Otin, C. Deubiquitinases in cancer: new functions and therapeutic options. Oncogene 31, 2373-2388 (2012).
26. Ovaa, H., Galardy, P. J. & Ploegh, H. L. Mechanism-based proteomics tools based on ubiquitin and ubiquitin-like proteins: synthesis of active site-directed probes. Methods in enzymology 399, 468-478 (2005).
27. D'Arcy, P. et al Inhibition of proteasome deubiquitinating activity as a new cancer therapy. Nat Med 17, 1636-1640 (2011).
28. Lee, B. H. et al. USP14 deubiquitinates proteasome-bound substrates that are ubiquitinated at multiple sites. Nature 532, 398-401 (2016).
29. Tian, Z. et al. A novel small molecule inhibitor of deubiquitylating enzyme USP14 and UCHL5 induces apoptosis in multiple myeloma and overcomes bortezomib resistance. Blood 123, 706-716 (2014).
30. Nazarian, R. et al. Melanomas acquire resistance to B-RAF(V600E) inhibition by RTK or N-RAS upregulation. Nature 468, 973-977 (2010).
31. Hu, M. et al. Structure and mechanisms of the proteasome-associated deubiquitinating enzyme USP14. EMBO J 24, 3747-3756 (2005).
32. Peth, A., Besche, H. C. & Goldberg, A. L. Ubiquitinated proteins activate the proteasome by binding to Usp14/Ubp6, which causes 20S gate opening. Mol Cell 36, 794-804 (2009).
33. Lee, B. H. et al. Enhancement of proteasome activity by a small-molecule inhibitor of USP14. Nature 467, 179-184 (2010).
34. Vogel, R. I. et al. Simultaneous inhibition of deubiquitinating enzymes (DUBs) and autophagy synergistically kills breast cancer cells. Oncotarget 6, 4159-4170 (2015).
35. Cancer Genome Atlas, N. Genomic Classification of Cutaneous Melanoma. Cell 161, 1681-1696 (2015).
36. Widmer, D. S. et al. Systematic classification of melanoma cells by phenotype-specific gene expression mapping. Pigment Cell Melanoma Res 25, 343-353 (2012).
37. Wang, X. et al. The proteasome deubiquitinase inhibitor VLX1570 shows selectivity for ubiquitin-specific protease-14 and induces apoptosis of multiple myeloma cells. Sci Rep 6, 26979 (2016).
38. Paulus, A. et al. Coinhibition of the deubiquitinating enzymes, USP14 and UCHL5, with VLX1570 is lethal to ibrutinib- or bortezomib-resistant Waldenstrom macroglobulinemia tumor cells. Blood cancer journal 6, e492 (2016).
39. Sarhan, D. et al. A novel inhibitor of proteasome deubiquitinating activity renders tumor cells sensitive to TRAIL-mediated apoptosis by natural killer cells and T cells. Cancer immunology, immunotherapy: CII 62, 1359-1368 (2013).
40. Villadolid, J. & Amin, A. Immune checkpoint inhibitors in clinical practice: update on management of immune-related toxicities. Translational lung cancer research 4, 560-575 (2015).
41. Beck, D. et al. Vemurafenib potently induces endoplasmic reticulum stress-mediated apoptosis in BRAFV600E melanoma cells. Science signaling 6, raj (2013).
42. Ma, X. H. et al. Targeting ER stress-induced autophagy overcomes BRAF inhibitor resistance in melanoma. J Clin Invest 124, 1406-1417 (2014).
43. Cerezo, M. et al. Compounds Triggering ER Stress Exert Anti-Melanoma Effects and Overcome BRAF Inhibitor Resistance. Cancer Cell 29, 805-819 (2016).
44. Demirsoy, S., Martin, S., Maes, H. & Agostinis, P. Adapt, Recycle, and Move on: Proteostasis and Trafficking Mechanisms in Melanoma. Frontiers in oncology 6, 240 (2016).

1. A method for predicting the survival time of a subject suffering from melanoma and treating a subject having a short predicted survival time, comprising the steps of i) quantifying the expression level of USP14 in a biological sample obtained from the subject; ii) administering a therapeutically effective amount of an inhibitor of USP14 to the subject having a short predicted survival time, wherein the subject is identified as having a short survival time when the expression level of USP14 quantified in step i) is lower than a predetermined reference value.

2. A method for treating melanoma in a subject in need thereof comprising a step of administering to said subject a therapeutically effective amount of an inhibitor of USP14.

3. The method according to claim 2, wherein, the subject has a short predicted survival time.

4. The method according to claim 2, wherein, the inhibitor of USP14 is a small organic molecule.

5. The method according to claim 4 wherein, the small molecule is VLX1570.

6. The method according to claim 4 wherein, the small molecule is b-AP15.

7. The method according to claim 4, wherein, the small molecule is IU1.

8. The method according to claim 2 wherein, the inhibitor of USP14 is siRNA.

9. A method for treating resistant melanoma in a subject in need thereof comprising a step of administering to said subject a therapeutically effective amount of an inhibitor of USP14.

10. The method according to claim 9, wherein, the melanoma is resistant to a treatment with one or more inhibitors of BRAF mutations.

11. The method according to claim 9, wherein, the melanoma is resistant to a treatment with one or more inhibitors of MEK mutations.

12. The method according to claim 9, wherein, the melanoma is resistant to a treatment with one or more inhibitors of NRAS mutations.

13. The method according to claim 9, wherein, the melanoma is resistant to treatment with one or more inhibitors of double-negative BRAF and NRAS mutations.

14. The method according to claim 9, wherein, the melanoma is resistant to a treatment with an immune checkpoint inhibitor.

15. A method of selecting a test compound that is a drug suitable for the treatment of melanoma comprising
i) providing a test compound
ii) determining the ability of said test compound to inhibit the activity of USP14, and
iii) selectin, the test compound as a drug when the test compound inhibits USP14.
